(19) United States
(12) Patent Application Publication
(10) Pub. No.: US 2024/0075012 A1
(43) Pub. Date: Mar. 7, 2024

(54) COMPOSITIONS AND METHODS FOR TREATING VIRAL INFECTION

(71) Applicant: President and Fellows of Harvard College, Cambridge, MA (US)

(72) Inventors: Haiqing Bai, Cambridge, MA (US); Longlong Si, Cambridge, MA (US); Rachelle Prantil-Baun, Cambridge, MA (US); Donald E. Ingber, Cambridge, MA (US)

(73) Assignee: President and Fellows of Harvard College, Cambridge, MA (US)

(21) Appl. No.: 18/448,881

(22) Filed: Aug. 11, 2023

Related U.S. Application Data
(63) Continuation of application No. 17/669,595, filed on Feb. 11, 2022, now abandoned.
Application No. 17/669,595 is a continuation of application No. PCT/US2022/011829, filed on Jan. 10, 2022.
(60) Provisional application No. 63/135,834, filed on Jan. 11, 2021.
(60) Provisional application No. 63/187,498, filed on May 12, 2021.

(51) Int. Cl. A61K 31/417; A61P 29/02; A61P 31/12

(52) U.S. Cl. CPC A61K 31/417; A61P 29/02; A61P 31/12

(57) ABSTRACT

The present disclosure provides compositions and methods for inhibiting respiratory viral infections, inflammatory diseases, and/or respiratory inflammation.

### RELATED APPLICATIONS

This application claims the benefit under 35 U.S.C. § 119(e) of U.S. provisional application Ser. No. 63/135,834, filed Jan. 11, 2021, and U.S. provisional application Ser. No. 63/187,498, filed May 12, 2021, each of which is incorporated by reference herein in its entirety.

### GOVERNMENT LICENSE RIGHTS

This invention was made with government support under HL141797 awarded by National Institutes of Health and HR0011-20-2-0040 awarded by the Department of Defense/DARPA. The government has certain rights in the invention.

### BACKGROUND

Respiratory viruses are the most frequent causative agents of disease in humans, impacting morbidity and mortality worldwide, and many of their injurious effects are due to stimulation of inflammatory responses in host tissues and organs. Common respiratory agents from several virus families are well adapted to efficient person-to-person transmission and circulate globally. Community-based studies have confirmed that these viruses are the predominant etiological agents of acute respiratory infections. The respiratory viruses that most commonly circulate as endemic or epidemic agents are influenza virus, respiratory syncytial virus, parainfluenza viruses, metapneumovirus, rhinovirus, coronaviruses, adenoviruses, and bocaviruses. Vaccines and effective antiviral drugs are not yet available for most of these viruses.

### SUMMARY

The present disclosure provides, in some aspects, compositions and methods for inhibiting inflammatory response to infection of a virus, for example, a respiratory virus, such as an influenza virus or a coronavirus, and thereby decreasing disease morbidity and mortality. The compositions and methods also may synergize with antiviral drugs or vaccines to further inhibit viral infection and replication. Respiratory viruses, including influenza viruses and coronaviruses, pose great challenge for public health. While antiviral agents and vaccines directly target viruses, their development usually lags behand the progress of pandemic, and their effects are compromised by rapid viral evolution. Regardless of the nature of the viruses, they usually only cause severe symptoms when they stimulate host inflammatory responses, and this can be augmented by the spread of infection from the upper airway to the distal lung, causing viral pneumonia, lung edema, and acute respiratory distress syndrome (ARDS). The spread of infection to other organs can also cause severe symptoms due to inflammation at these distant sites.

Induction of an aberrant host inflammatory response is the decisive factor that differentiates between mild symptom or severe symptom. Therefore, agents that are able to tamper down the host immune response may represent a universal treatment for all infectious disease, such as respiratory infectious diseases. For example, dexamethasone, a corticosteroid that has anti-inflammatory effect, is the first and only drug to date that demonstrates clinical benefits against patients with late-stage coronavirus disease 2019 (COVID-19), however, it is only effective when administered during late stages of the disease (Tomazini, B. M. et al. JAMA: The journal of the American Medical Association 324, 1307-1316, doi:10.1001/jama.2020.17021 (2020)).

The data provided herein advance an understanding of signaling pathways that drive hyperinflammatory states following viral infection, such as respiratory virus infection, for example, in the distal lung alveolus. Further, the data herein demonstrate that inhibitors of a particular pathway, the S100/RAGE pathway, can be used to suppress viral-induced inflammatory responses. Thus, such inhibitors may be used as a broad-spectrum treatment of viral infection, for example, respiratory viral infection. In addition, the data show that inhibitors of the S100/RAGE pathway can synergize with antiviral drugs to increase suppression of viral replication in addition to inhibiting inflammation.

Some aspects of the present disclosure provide methods of treating a viral infection, such as a respiratory virus infection, in a subject in need thereof, comprising administering to the subject a receptor for advanced glycation end-products (RAGE) pathway inhibitor. In some embodiments, the subject is infected with a virus, such as a respiratory virus. In some embodiments, the subject is at risk of a viral infection, such as a respiratory virus infection.

The present disclosure also contemplates the use of RAGE pathway inhibitors (alone or in combination with an antiviral agent) to treat an inflammatory response by a host caused by a disease state, including infections caused by viruses. It should be understood that the RAGE pathway inhibitors provided herein may be used to treat diseases, for example, those caused by viral infection, such as respiratory virus infection, by suppressing the host inflammatory response, which decreases morbidity and death. When combined with antiviral agents, RAGE pathway inhibitors may also augment inhibition of viral infection itself.

In some embodiments, the inflammation triggered by a viral infection, such as a respiratory virus infection, is located in the lung. In other embodiments, the inflammation triggered by a viral infection, such as a respiratory virus infection, is located in organs other than the lung. Thus, the present disclosure contemplates methods of treating a viral infection that include suppressing the host inflammatory response in the lung and/or organs other than the lung.

In some embodiments, the respiratory virus is selected from the group consisting of influenza viruses, or influenza A/Avian Influenza (H5N1)), coronaviruses, rhinoviruses, enteroviruses, parainfluenza viruses, metapneumoviruses, respiratory syncytial viruses, adenoviruses, and bocaviruses. For example, the influenza viruses may be influenza A/Hong Kong/8/68 (H3N2) or A/WSN/33 (H1N1). For example, the coronaviruses may be betacoronavirus such as MERS-CoV, SARS-CoV, SARS-CoV-2, or a common cold virus, such as OC43.

In some embodiments, the subject has one or more symptom(s) of a viral infection.

In some embodiments, the symptom is aberrant inflammation.

In some embodiments, the symptom is inflammation in the lungs. In other embodiments, the symptom is inflammation in an organ other than the lungs.

In some embodiments, the RAGE pathway inhibitor inhibits RAGE signaling. In some embodiments, the RAGE pathway inhibitor is an inhibitor of RAGE gene expression, mRNA expression, protein expression, and/or protein activity (e.g., signaling).

In some embodiments, the RAGE pathway inhibitor inhibits expression and/or activity of a S100 family member. In some embodiments, the RAGE pathway inhibitor inhibits binding of the S100 family member to RAGE. In some embodiments, the RAGE pathway inhibitor competes with the S100 family member for binding to RAGE.

In some embodiments, the S100 family member is selected from the group consisting of S100A7, S100A7A, S100A6, S100A8, S100A9, and S100A12. For example, the S100 family member may be S100A7.

In some embodiments, the RAGE pathway inhibitor is a chemical compound. In some embodiments, the chemical compound is selected from the group consisting of azeliragon, FPS-ZM1, 4,6-bisphenyl-2-(3-alkoxyanilino) pyrimidine, and pyrazole-5-carboxamides.

In some embodiments, the RAGE pathway inhibitor is a RAGE-antagonist peptide (RAP). In some embodiments, the RAGE-antagonist peptide is selected from the group consisting of S100P-derived RAPs and high mobility group box-1 (HMGB-1)-derived RAPs.

In some embodiments, the RAGE pathway inhibitor is an antisense oligonucleotide.

In some embodiments, the RAGE pathway inhibitor is an RNA interference molecule. In some embodiments, the RNA interference molecule is selected from the group consisting of micro RNAs, short interfering RNAs, and short hairpin RNAs.

In some embodiments, the RAGE pathway inhibitor is soluble RAGE.

In some embodiments, the RAGE pathway inhibitor is a programmable nuclease. For example, the programmable nuclease may be an RNA-guided nuclease. In some embodiments, the programmable nuclease is selected from the group consisting of CRISPR nucleases, zinc finger nucleases, transcription activator-like effector nucleases, and meganucleases.

In some embodiments, the RAGE pathway inhibitor is administered in an effective amount to suppress inflammation associated with respiratory viral infection (e.g., inflammation in the lung and/or other organs). In some embodiments, an antiviral agent is administered to the subject. In some embodiments, the antiviral agent is favipiravir. In some embodiments, the antiviral agent is molnupiravir. In some embodiments, both azeliragon and favipiravir are administered to the subject (e.g., independently or formulated together). In some embodiments, both azeliragon and molnupiravir are administered to the subject (e.g., independently or formulated together).

In some embodiments, an inducer of host protective response is administered to the subject. For example, the inducer of host protective response may be a type I interferon. In some embodiments, both azeliragon and the inducer of host protective response (e.g., a type I interferon) are administered to the subject (e.g., independently or formulated together).

Further aspects of the present disclosure provide methods of treating acute respiratory distress syndrome (ARDS) in a subject in need thereof, comprising administering to the subject a RAGE pathway inhibitor. In some embodiments, the subject is diagnosed with ARDS.

In some embodiments, the subject requires use of a respiratory ventilator.

Still other aspects of the present disclosure provide methods of treating pulmonary barotrauma, such as ventilator-induced lung injury in a subject in need thereof, comprising administering to the subject a RAGE pathway inhibitor, wherein the subject is undergoing treatment with a ventilator or has an injury induced by treatment with a ventilator. In some embodiments, administration of the RAGE pathway inhibitor reduces inflammation of the lung caused by treatment with a ventilator or other forms of barotrauma.

Yet other aspects of the present disclosure provide methods of treating an inflammatory disease, such as an inflammatory lung disease, in a subject in need thereof, comprising administering to the subject a RAGE pathway inhibitor. In some embodiments, the subject is diagnosed with an inflammatory lung disease.

In some embodiments, the inflammatory lung disease is a chronic inflammatory lung disease. In some embodiments, the chronic inflammatory lung disease is Chronic Obstructive Pulmonary Disease (COPD).

Some aspects of the present disclosure provide a method of preventing viral disease-associated inflammation in a subject in need thereof, comprising administering to the subject a RAGE pathway inhibitor (prior to infection with a virus).

Other aspects of the present disclosure provide a method of treating viral disease-associated inflammation in a subject in need thereof, comprising administering to the subject a RAGE pathway inhibitor, wherein the subject has been infected with a virus.

In some embodiments, the subject has symptoms of viral disease.

In some embodiments, the RAGE pathway inhibitor is administered with an antiviral agent, such as molnupiravir or favipiravir.

### BRIEF DESCRIPTION OF THE DRAWINGS

FIGS. 1A-1C. Breathing motions inhibit influenza infection. immunostaining of influenza nuclear protein (NP) in Alveolar Chips that were cultured under either static condition (left image) or under 5% and 0.25 Hz cyclic mechanical strains (right image) for 48 hours and then infected with H3N2 at MOI (multiplicity of infection)=1 for another 48 hours (FIG. 1A). qPCR quantification of viral RNA in the epithelial cell lysates after the treatment under static condition or under 5% and 0.25 Hz cyclic mechanical strains (FIG. 1B). Viral titers from the apical washes after the treatment under static condition or under 5% and 0.25 Hz cyclic mechanical strains (FIG. 1C).

FIGS. 2A-2E. S100A7 responses to mechanical stimuli. Volcano plot of alveolar epithelial cells under static condition or under 5% mechanical strains. S100A7 is a top upregulated gene in the strain condition (FIG. 2A). Increased S100A7 during Human Alveolus Chip culture on day 14 after the treatment under 5% mechanical strains. ANOVA with paired t test, *** p<0.001 (FIG. 2B). Mechanical strain induced the expression of S100A7 in epithelial cells (left) and in endothelial cells (right) in the Human Alveolus Chip model. 0%=static; 0.5%=physiological strain; 10%=hyperphysiological strain (FIG. 2C). ELISA for S100A7 in the Alveolus Chip apical washes 4 days after static culture or under mechanical strains. Student's t test, **p<0.01 (FIG. 2D). Decreased S100A7 expression after stopping mechanical strains for 4 days compared with 5% or 10% strains. ANOVA with paired t test, *** p<0.01 (FIG. 2E).

FIGS. 3A-3B. S100A7 family genes are induced by viral infection. Volcano plot of RNA-seq results from epithelial cells in Human Alveolus Chips infected with H3N2 at MOI=1 for 48 hours. Members of the S100 family genes that are differentially expressed are labeled (FIG. 3A). Volcano plot of RNA-seq that was resulted from epithelial cells in Human Alveolus Chips infected with OC43 at MOI=5 for 48 hours (FIG. 3B).

FIGS. 4A-4B. S100A7 was increased in COPD (chronic obstructive pulmonary disease). Volcano plot for RNA-seq of human airway epithelial cells from normal donors or patients with COPD (FIG. 4A). Increased S100A7 expression in COPD patients. Mann Whitney test, ****p<0.0001 (FIG. 4B).

FIGS. 5A-5B. RAGE pathway inhibitor azeliragon blocked viral cytokine responses. 100 nM azeliragon was added 24 hours before infection of H3N2 at MOI=1. Cytokines (IL-6, IL-8, or IP-10 (FIG. 5A), or MCP-1, RANTES, or IL-29 (FIG. 5B)) were measured using Luminex kit at 48 hours post infection.

FIG. 5C. RAGE pathway inhibitor azeliragon blocked viral cytokine responses. 20 nM azeliragon was added 24 hours before infection of H3N2 at MOI=1. Cytokines (IL-6, IL-8, IP-10, and Granulocyte-macrophage colony-stimulating factor (GM-CSF)) and S100A8/9 were measured using Luminex kit at 48 hours post infection (FIG. 5C).

FIG. 6. RAGE pathway inhibitor azeliragon inhibits cyclic force-induced inflammation (biotrauma). DMSO control or 100 nM azeliragon were perfused for 48 hours in the presence of 0% strain, 5% strain, or 10% strain; vascular outlet was collected for a Luminex assay.

FIGS. 7A-7B. RAGE pathway inhibitor FPS-ZM1 blocked viral cytokine responses. 200 nM FPS-ZM1 was added 24 hours before infection of H3N2 at MOI=1. Cytokines (IL-18, IL-8, or IL-29 (FIG. 7A), or IL-6, IP-10, or RANTES (FIG. 7B)) were measured using Luminex kit at 48 hours post infection.

FIGS. 8A-8C. RAGE pathway inhibitor combination azeliragon and molnupiravir blocked viral cytokine responses. 100 nM azeliragon and 0.5 μM molnupiravir were added 2 hours before infection of H3N2 at MOI=0.01. Cytokines (NP, IL-6, or CXCL-10 (FIG. 8A), or IL-18, IP-6, or IL-8 (FIG. 8B), or RANTES, TNFα, or CXCL-10 (FIG. 8C)) were measured using Luminex kit at 48 hours post infection.

FIGS. 9A-9B. RAGE pathway inhibitor combination azeliragon and favipiravir blocked viral cytokine responses. 100 nM azeliragon and 500 nM favipiravir was added 2 hours before infection of H3N2 at MOI=1. The combination decreased viral load, as assessed from apical washes (left) and epithelial cell lysates (right) (FIG. 9A). The combination also decreased inflammatory cytokine production (IL-6, CXCL10, and CCLS) (FIG. 9B) as assessed from epithelial cell lysates using a Luminex assay at 48 hours post infection.

### DETAILED DESCRIPTION

Provided herein, in some aspects, are methods for using RAGE pathway inhibitors, such as azeliragon (also referred to as TTP488), to suppress inflammation associated with viral infection, for example, by various types of respiratory viruses, including various influenza and coronavirus strains (e.g., SARS-CoV-2). In some embodiments, the inflammation associated with infection is in the lung. In some embodiments, the inflammation associated with infection is in an organ other than the lung.

Other RAGE pathway inhibitors (e.g., FPS-ZM1 and/or azeliragon derivatives) may also be effective, in some instances. In some embodiments, soluble RAGE, siRNA or CRISPR Cas9 against RAGE may also be used. These drugs may be used in combination with antivirals, such as molnupiravir (also referred to as the EIDD 2801 prodrug or is its active metabolite EIDD 1931) or favipiravir, for example, as well as inducers of host protective responses (e.g., type I interferons).

Further, S100A7, for example, is a top upregulated gene in COPD patients, thus, in some embodiments, it can be used as a biomarker for COPD and inhibitors of the S100A7/RAGE pathway may be used as a treatment to suppress inflammation in COPD patients.

As inflammation and ARDS are observed in patients on respiratory ventilators that exert cyclic mechanical strain on lung, RAGE pathway inhibitors may also be useful, in some aspects, to suppress inflammation in these conditions.

### RAGE Pathway Inhibition

The multiligand receptor for advanced glycation end products (RAGE) of the immunoglobulin superfamily is expressed on multiple cell types implicated in the immune-inflammatory response and in atherosclerosis. RAGE is found in human airways with high basal levels of RAGE expressed in pulmonary tissue. Specifically, RAGE is expressed at the highest levels in the lung compared to other tissues, in particular in alveolar type I cells.

The receptor is membrane bound and is also known as full length RAGE (flRAGE) or membrane RAGE (mRAGE). Ligand-RAGE interaction on cells, such as monocytes, macrophages, and endothelial cells, mediates cellular migration and upregulation of proinflammatory and prothrombotic molecules.

Without wishing to be bound by any theory, RAGE binds a broad range of ligands associated with inflammatory responses, including advanced glycation end products (AGE), β-sheet fibrillary structures β-amyloid & serum amyloid A), amphoterin (HMGB1), members of the S100/calgranulin family such as S100A12, Mac-1, and phosphatidylserine.

RAGE expression and its signaling can be regulated both by its ligands and by RAGE isoforms known collectively as soluble RAGE (sRAGE). sRAGE contains the extracellular domain of RAGE and can bind to circulating pro-inflammatory ligands preventing their binding to mRAGE thereby preventing RAGE activation. sRAGE includes a combination of isoforms that are generated in at least two ways: 1) cleaved RAGE (cRAGE) which results from the proteolytic cleavage of mRAGE (ectodomain shedding) from the cell membrane, and 2) alternative splicing of the RAGE transcript resulting in 10 variants detected in the human lung. Of these the most significant is an endogenous soluble RAGE (esRAGE). Importantly decreased levels of esRAGE and/or increases in mRAGE are thought to enhance RAGE mediated inflammation.

Provided herein, in some aspects, are method of treating a viral infection, such as a respiratory virus infection in a subject, comprising administering to a subject a receptor for advanced glycation end-products (RAGE) pathway inhibitor, wherein the subject is infected with or at risk of viral infection. A “RAGE pathway inhibitor” is an agent that reduces a measurable level of or eliminates signaling through the RAGE pathway, for example, by directly inhibiting RAGE signaling or signaling of another member of the RAGE pathway. In some embodiments, the RAGE pathway is a S100/RAGE pathway. In some embodiments, signaling is reduced by at least 30%. For example, a RAGE pathway inhibitor may reduce RAGE pathway signaling by at least 35%, at least 40%, at least 45%, at least 50%, at least 55%, at least 60%, at least 65%, at least 70%, at least 75%, at least 80%, at least 85%, at least 90%, at least 95%, or 100%, relative to a control. A control may be, for example, a measurable level of RAGE pathway signaling in the absence of the RAGE pathway inhibitor. A level of RAGE pathway signaling may be determined, for example, by known or later-developed methods for measuring protein signaling of members in the RAGE pathway. See, e.g., Haslbeck K M et al. Neurol Res. 2007 Jan;29(1):103-10; Ramasamy R et al. Ann N Y Acad Sci. 2011 December; 1243: 88-102; and Senatus L M 2017 Dec. 5; 8:187.

In some embodiments, a RAGE pathway inhibitor inhibits RAGE signaling. For example, a RAGE pathway inhibitor may reduce RAGE signaling by at least 35%, at least 40%, at least 45%, at least 50%, at least 55%, at least 60%, at least 65%, at least 70%, at least 75%, at least 80%, at least 85%, at least 90%, at least 95%, or 100%, relative to a control. A control may be, for example, a measurable level of RAGE signaling in the absence of the RAGE pathway inhibitor.

Inhibition of RAGE signaling may be achieved, in some embodiments, by inhibiting RAGE gene expression, mRNA expression, protein expression, and/or protein activity (e.g., signaling). In some embodiments, the RAGE pathway inhibitor is an inhibitor of RAGE gene expression. In other embodiments, the RAGE pathway inhibitor is an inhibitor of RAGE mRNA expression. In yet other embodiments, the RAGE pathway inhibitor is an inhibitor of RAGE protein expression. In still other embodiments, the RAGE pathway inhibitor is an inhibitor of RAGE protein activity (e.g., signaling). In some embodiments, the RAGE pathway inhibitor is an inhibitor of any combination of RAGE gene expression, mRNA expression, protein expression, and protein activity. In some embodiments, the RAGE pathway inhibitor is any inhibitor that can inhibit any aspects of the RAGE pathway so that the signaling is blocked.

In some embodiments, the RAGE pathway inhibitor inhibits expression and/or activity of a S100 family member. For example, a RAGE pathway inhibitor may inhibit binding of the S100 family member to RAGE. In some embodiments, the RAGE pathway inhibitor competes with the S100 family member for binding to RAGE.

The S100 protein family is composed of 21 members that exhibit a high degree of structural similarity but are not functionally interchangeable. The S100 proteins possess two calcium-binding sites that have helix-loop-helix (“EF-hand type”) conformation. This family of proteins modulates cellular responses by acting both as intracellular Ca2+ sensors and as extracellular factors that bins to various membrane receptors. S100 proteins are present in cells derived from the neural crest such as Schwann cells and melanocytes, chondrocytes, adipocytes, myoepithelial cells, macrophages, Langerhans cells, dendritic cells, keratinocytes, or breast epithelial cells. S100 protein family includes genes such as S100A1, S100A2, S100A3, S100A4, S100A5, S100A6, S100A7 (psoriasin), S100A8 (calgranulin A), S100A9 (calgranulin B), S100A10, S100A11, S100A12 (calgranulin C), S100A13, S100A14, S100A15 (koebnerisin), S100A16, S100B, S100P, and S100Z. S100A7, along with S100A6, S100A8, S100A9, and S100A12, bind to the receptor for advanced glycation end-products (RAGE) and thereby, promote inflammation.

In some embodiments, the S100 family member as disclosed herein is selected from S100A7, S100A7A, S100A6, S100A8, S100A9, and S100A12. In some embodiments, the S100 family member is S100A7. In some embodiments, the S100 family member is any suitable S100 protein that may bind to RAGE and trigger the RAGE pathway.

In some embodiments, the RAGE pathway inhibitor is a chemical compound. In some embodiments, the chemical compound is selected from the group consisting of: azeliragon; FPS-ZM1; 4,6-bisphenyl-2-(3-alkoxyanilino)pyrimidine; and pyrazole-5-carboxamides. In some embodiments, the chemical compound is azeliragon. In other embodiments, the chemical compound is FPS-ZM1. In yet other embodiments, the chemical compound is 4,6-bisphenyl-2-(3-alkoxyanilino) pyrimidine. In still other embodiments, the chemical compound is pyrazole-5-carboxamides.

In some embodiments, a therapeutically effective amount of azeliragon is administered. The therapeutically effective amount of azeliragon can be a dose of 10-100 nM, 10-50 nM, 20-100 nM, 20-50 nM, 50 nM-500 nM, 50 nM-450 nM, 50 nM-400 nM, 50 nM-350 nM, 50 nM-300 nM, 50 nM-250 nM, 50 nM-200 nM, 50 nM-150 nM, 50 nM-100 nM, 50 nM-75 nM, 75 nM-400 nM, 75 nM-350 nM, 75 nM-300 nM, 75 nM-250 nM, 75 nM-200 nM, or 75 nM-150 nM, for example. The therapeutically effective amount of azeliragon can be a dose of 20 nM or 100 nM.

In some embodiments, the RAGE pathway inhibitor is a RAGE-antagonist peptide (RAP). For example, the RAP may be selected from the group consisting of: S100P-derived RAPs (e.g., Arumugam T et al. Clin Cancer Res. 2012 Aug. 15; 18(16):4356-64) and high mobility group box-1 (HMGB-1)-derived RAPs (e.g., Ulloa L et al. Cytokine Growth Factor Rev. 2006 June;17(3):189-201).

In some embodiments, the RAGE pathway inhibitor is an antisense oligonucleotide. Antisense oligonucleotide (ASOs) are small-sized single-stranded nucleic acids that bind to their target RNA or DNA sequence inside cells to cause gene silencing. In some embodiments, a RAGE pathway inhibitor is an ASO that binds to a nucleic acid encoding RAGE or a RAGE pathway member, e.g., an S100 family member, such as S100A7, S100A7A, S100A6, S100A8, S100A9, and/or S100A12.

In other embodiments, the RAGE pathway inhibitor is an RNA interference molecule. Non-limiting examples of RNA interference molecules include micro RNAs, short interfering RNAs, and short hairpin RNAs. Small RNA molecules regulate eukaryotic gene expression during development and in response to stresses including viral infection. Specialized ribonucleases and RNA binding proteins govern the production and action of small regulatory RNAs. After initial processing in the nucleus by Drosha, pre-miRNAs are transported to the cytoplasm, where Dicer cleavage generates mature microRNAs (miRNAs) and short interfering RNAs (siRNAs). These double-stranded products assemble with Argonaute proteins such that one strand is preferentially selected and used to guide sequence-specific silencing of complementary target mRNAs by endonucleolytic cleavage or translational repression. See, e.g., Wilson R et al. Annu Rev Biophys. 2013; 42: 217-239). In some embodiments, a RAGE pathway inhibitor is an RNA interference molecule that binds to a nucleic acid encoding RAGE or a RAGE pathway member, e.g., an S100 family member, such as S100A7, S100A7A, S100A6, S100A8, S100A9, and/or S100A12.

In some embodiments, the RAGE pathway inhibitor is soluble RAGE. As discussed above, sRAGE contains the extracellular domain of RAGE and can bind to circulating pro-inflammatory ligands preventing their binding to mRAGE thereby preventing RAGE activation.

In some embodiments, the RAGE pathway inhibitor is a programmable nuclease, for example, an RNA-guided nuclease. Non-limiting examples of programmable nucleases include CRISPR nucleases, zinc finger nucleases, transcription activator-like effector nucleases, and meganucleases.

Transcription activator-like effector nucleases (TALEN) are restriction enzymes that can be engineered to cut specific sequences of DNA. They are made by fusing a TAL effector DNA-binding domain to a DNA cleavage domain (a nuclease which cuts DNA strands). Transcription activator-like effectors (TALEs) can be engineered to bind to practically any desired DNA sequence, so when combined with a nuclease, DNA can be cut at specific locations.[1] The restriction enzymes can be introduced into cells, for use in gene editing or for genome editing in situ, a technique known as genome editing with engineered nucleases.

Zinc-finger nucleases (ZFNs) are artificial restriction enzymes generated by fusing a zinc finger DNA-binding domain to a DNA-cleavage domain. Zinc finger domains can be engineered to target specific desired DNA sequences, and this enables zinc-finger nucleases to target unique sequences within complex genomes. By taking advantage of endogenous DNA repair machinery, these reagents can be used to precisely alter the genomes of higher organisms.

The CRISPR-Cas system is a prokaryotic immune system that confers resistance to foreign genetic elements such as those present within plasmids and phages and provides a form of acquired immunity. RNA harboring the spacer sequence helps Cas (CRISPR-associated) proteins recognize and cut foreign pathogenic DNA. Other RNA-guided Cas proteins cut foreign RNA. CRISPR are found in approximately 50% of sequenced bacterial genomes and nearly 90% of sequenced archaea. These systems have created CRISPR gene editing that commonly utilizes the cas9 gene.

For a review of ZFN, TALEN and CRISPR/Cas-based methods for genome engineering see, e.g., Gaj T et al. Trends Biotechnol. 2013 July; 31(7): 397-405, incorporated herein by reference.

Meganucleases are endodeoxyribonucleases characterized by a large recognition site (double-stranded DNA sequences of 12 to 40 base pairs); as a result, this site generally occurs only once in any given genome. For example, the 18-base pair sequence recognized by the I-SceI meganuclease would on average require a genome twenty times the size of the human genome to be found once by chance (although sequences with a single mismatch occur about three times per human-sized genome). Meganucleases are therefore considered to be the most specific naturally occurring restriction enzymes.

In some embodiments, a RAGE pathway inhibitor is a programmable nuclease system designed to target a nucleic acid encoding RAGE or a RAGE pathway member, e.g., an S100 family member, such as S100A7, S100A7A, S100A6, S100A8, S100A9, and/or S100A12.

Any combination of two or more of the agents provided herein may be administered to a subject to treat a viral infection, such as a respiratory virus infection. In some embodiments, any therapeutically effective amount of an agent as disclosed herein can be administered to a subject in need thereof.

### Pharmaceutical Compositions

In some aspects, the present disclosure provides compositions comprising any of the agents as disclosed herein. In some embodiments, the compositions further comprise a pharmaceutically acceptable excipient (e.g., carrier, buffer, and/or salt, etc.). A molecule or other substance/agent is considered “pharmaceutically acceptable” if it is approved or approvable by a regulatory agency of the Federal government or a state government or listed in the U.S. Pharmacopeia or other generally recognized pharmacopeia for use in animals, including humans. An excipient may be any inert (inactive), non-toxic agent, administered in combination with an agent provided herein. Non-limiting examples of pharmaceutically acceptable excipients include water, saline, dextrose, glycerol, ethanol and combinations thereof. The excipient may be selected on the basis of the mode and route of administration, and standard pharmaceutical practice.

Agents as disclosed herein, in some embodiments, may be formulated in a delivery vehicle. Non-limiting examples of delivery vehicles include nanoparticles, such as nanocapsules and nanospheres. See, e.g., Sing, R et al. Exp Mol Pathol. 2009; 86(3):215-223. In some embodiments, nanoparticles are less than 1 μm in diameter. In some embodiments, nanoparticles are between about 1 and 100 nm in diameter. Nanoparticles include organic nanoparticles, such as dendrimers, liposomes, or polymeric nanoparticles. Nanoparticles also include inorganic nanoparticles, such as fullerenes, quantum dots, and gold nanoparticles. Compositions may comprise an aggregate of nanoparticles. In some embodiments, the aggregate of nanoparticles is homogeneous, while in other embodiments the aggregate of nanoparticles is heterogeneous. A nanocapsule is often comprised of a polymeric shell encapsulating a drug (e.g., agents of the present disclosure). Nanospheres are often comprised of a solid polymeric matrix throughout which the drug (e.g., agent) is dispersed. In some embodiments, the nanoparticle is a lipid particle, such as a liposome. See, e.g., Puri, A et al. Crit Rev Ther Drug Carrier Syst. 2009; 26(6):523-80. The term ‘nanoparticle’ also encompasses microparticles, such as microcapsules and micro spheres.

Methods developed for making particles for delivery of encapsulated agents are described in the literature (for example, please see Doubrow, M., Ed., “Microcapsules and Nanoparticles in Medicine and Pharmacy,” CRC Press, Boca Raton, 1992; Mathiowitz and Langer, J. Controlled Release 5:13-22, 1987; Mathiowitz et al. Reactive Polymers 6:275-283,1987; Mathiowitz et al. J. Appl. Polymer Sci. 35:755-774, 1988; each of which is incorporated herein by reference).

General considerations in the formulation and/or manufacture of pharmaceutical agents, such as compositions comprising any of the agents disclosed herein may be found, for example, in Remington's Pharmaceutical Sciences, 18th edition, Mack Publishing Co., Easton, Pa (1990) (incorporated herein by reference in its entirety).

Although the descriptions of pharmaceutical compositions provided in this application are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

### Treatment Methods

Any of the agents or compositions disclosed herein may be administered to a subject (e.g., mammalian subject, such as a human, mouse, rabbit, goat, sheep or pig) to treat a viral infection, such as a respiratory virus infection. “Treatment” as used herein refers to the treatment of a disease (e.g., a disease caused by a viral infection), including the alleviation of one or more symptoms associated with the disease. Thus, “treating a viral infection,” including “treating a respiratory virus infection,” encompasses treating a disease caused by a viral infection, such as a respiratory virus infection.

Suitable routes of administration include, without limitation, intravenous, intranasal, intramuscular, subcutaneous, and inhalation. In some embodiments, an agent of the disclosure is administered intravenously, subcutaneous, intramuscularly or intranasally. In some embodiments, an agent of the disclosure is delivered to the lung, for example, via aerosol, nebulizer, or tracheal wash. Other routes of administration are contemplated herein. The administration route of an agent of the disclosure can be changed depending on a number of factors, including the pathogen and/or mechanism of pathogenesis. The route of administration of the compositions provided herein may vary depending on the specific agents (e.g., RAGE pathway inhibitor as a chemical compound, a programmable nuclease, or a small molecule).

In some embodiments, an effective amount (or therapeutically effective amount) of a RAGE pathway inhibitor of the present disclosure is administered to a subject to inhibit pathogenesis of a virus (e.g., respiratory virus). In some embodiments, an effective amount of a RAGE pathway inhibitor of the present disclosure is administered to a subject to suppress inflammation associated with viral infection, a such as respiratory viral infection (e.g., inflammation in the lung and/or other organ). In some embodiments, an effective amount of a RAGE pathway inhibitor is administered to a subject to alleviate one or more symptom associated with a disease caused by a viral infection. A therapeutically effective amount, in some embodiments, is an amount of a RAGE pathway inhibitor required to prevent viral infection or a disease caused by viral infection in a subject. In some embodiments, an effective amount is an amount of a RAGE pathway inhibitor required to prevent or reduce viral propagation in a subject. In some embodiments, an effective amount is an amount of a RAGE pathway inhibitor required to prevent or reduce viral survival (e.g., length of time a virus survives in a subject). In some embodiments, an effective amount is an amount of a RAGE pathway inhibitor required to reduce viral titer in a subject.

Effective amounts vary, as recognized by those skilled in the art, depending on the route of administration, excipient usage, and co-usage with other active agents. Effective amounts depend on the subject, including, for example, the weight, sex and age of the subject as well as the strength of the subject's immune system and/or genetic predisposition. Suitable dosage ranges are readily determinable by one skilled in the art.

The compositions herein may be administered as a single dose or as multiple doses (e.g., a booster dose or multiple booster doses). In certain embodiments, when multiple doses are administered to a subject, any two doses of the multiple doses include different or substantially the same amounts of a protein described in this application. Dosage forms may be administered at a variety of frequencies. In certain embodiments, when multiple doses are administered to a subject, the frequency of administering the multiple doses to the subject is three doses a day, two doses a day, one dose a day, one dose every other day, one dose every third day, one dose every week, one dose every two weeks, one dose every three weeks, or one dose every four weeks, or less frequent than every four weeks. In certain embodiments, the frequency of administering the multiple doses to the subject is one dose per day. In certain embodiments, the frequency of administering the multiple doses to the subject is two doses per day. In certain embodiments, the frequency of administering the multiple doses to the subject is three doses per day. In certain embodiments, when multiple doses are administered to a subject, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject. In certain embodiments, the duration between the first dose and last dose of the multiple doses is three months, six months, or one year. In certain embodiments, the duration between the first dose and last dose of the multiple doses is the lifetime of the subject. In some embodiments, dose ranging studies can be conducted to establish optimal therapeutic or effective amounts of the component(s) (e.g., proteins or peptides) to be present in dosage forms. In embodiments, the component(s) are present in dosage forms in an amount effective as a therapeutic intervention after diagnosis of viral infection, ARDS, or an inflammatory lung disease. In some embodiments, a composition is administered as a prophylactic treatment before diagnosis of viral infection, ARDS, or an inflammatory lung disease.

In some embodiments, more than one agents associated with the disclosure is administered to a subject. In some embodiments, the agents are administered concomitantly. In other embodiments, the agents are not administered concomitantly. In some embodiments, the first agent is not administered within 1 month, 1 week, 6 days, 5, days, 4 days, 3 days, 2 days, 1 day, 12 hour, 6 hours, 5 hours, 4 hours, 3 hours, 2 hours, or 1 hour of the second agent.

The term “concomitantly” refers to administering two or more agents to a subject in a manner that is correlated in time, preferably sufficiently correlated in time such that a first agent has an effect on a second agent, such as increasing the efficacy of the second agent, preferably the two or more agents are administered in combination. In some instance, a second agent has an effect on a first agent, such as regulating the efficacy of the first composition. In some embodiments, concomitant administration may encompass administration of two or more agents within a specified period of time. In some embodiments, the two or more agents are administered within 1 month, within 1 week, within 1 day, or within 1 hour. In some embodiments, concomitant administration encompasses simultaneous administration of two or more agents. In some embodiments, when two or more agents are not administered concomitantly, there is little to no effect of the first agent on the second agent.

The compositions provided herein may include, or may be administered in combination with, other agents, such as antiviral agents, to the subject. The compositions provided herein may include, or may be administered in combination with, other agents, such as an inducer of host protective response, to the subject. In some embodiments, an inducer of host protective response can be a type I interferon, for example. In some embodiments, an inducer of host protective response can be any compound, agent, or substance that is capable of inducing host protective immune response. Such compound, agent, or substance can include but are not limited to chemokines.

In some embodiments, an agent, or a combination of agents, as disclosed herein, is administered in an amount effective for decreasing viral infectivity, such as respiratory virus infectivity. In some embodiments, viral infectivity, such as respiratory virus infectivity, is decreased by at least 20%, relative to a control. For example, viral infectivity, such as respiratory virus infectivity, may be decreased by at least 30%, at least 40%, at least 50%, at least 60%, at least 70%, at least 80%, or at least 90%, relative to a control. In some embodiments, viral infectivity, such as respiratory virus infectivity, is decreased by 20%-100%, 20%-90%, 20%-80%, 20%-70%, 20%-60%, 20%-50%, 30%-100%, 30%-90%, 30%-80%, 30%-70%, 30%-60%, 30%-50%, 40%-100%, 40%-90%, 40%-80%, 40%-70%, 40%-60%, 40%-50%, 50%-100%, 50%-90%, 50%-80%, 50%-70%, or 50%-60%, relative to a control.

In some embodiments, an agent, or a combination of agents, is administered in an amount effective for increasing viral inhibition rate. In some embodiments, viral inhibition rate is increased by at least 20%, relative to a control. For example, viral infectivity, such as respiratory virus infectivity, may be increased by at least 30%, at least 40%, at least 50%, at least 60%, at least 70%, at least 80%, or at least 90%, relative to a control. In some embodiments, viral inhibition rate is increased by 20%-100%, 20%-90%, 20%-80%, 20%-70%, 20%-60%, 20%-50%, 30%-100%, 30%-90%, 30%-80%, 30%-70%, 30%-60%, 30%-50%, 40%-100%, 40%-90%, 40%-80%, 40%-70%, 40%-60%, 40%-50%, 50%-100%, 50%-90%, 50%-80%, 50%-70%, or 50%-60%, relative to a control.

In some embodiments, an agent, or a combination of agents, is administered in an amount effective for inhibiting binding of the S100 family member to RAGE. In some embodiments, the inhibition of the binding is increased by at least 20%, relative to a control. For example, the inhibition of the binding of the S100 family member to RAGE may be increased by at least 30%, at least 40%, at least 50%, at least 60%, at least 70%, at least 80%, or at least 90%, relative to a control. In some embodiments, the inhibition of the S100 family member to RAGE is increased by 20%-100%, 20%-90%, 20%-80%, 20%-70%, 20%-60%, 20%-50%, 30%-100%, 30%-90%, 30%-80%, 30%-70%, 30%-60%, 30%-50%, 40%-100%, 40%-90%, 40%-80%, 40%-70%, 40%-60%, 40%-50%, 50%-100%, 50%-90%, 50%-80%, 50%-70%, or 50%-60%, relative to a control.

In some embodiments, an agent, or a combination of agents, is administered in an amount effective for treating, e.g., improving the symptoms of acute respiratory distress syndrome (ARDS) by at least 20%-100%, 20%-90%, 20%-80%, 20%-70%, 20%-60%, 20%-50%, 30%-100%, 30%-90%, 30%-80%, 30%-70%, 30%-60%, 30%-50%, 40%-100%, 40%-90%, 40%-80%, 40%-70%, 40%-60%, 40%-50%, 50%-100%, 50%-90%, 50%-80%, 50%-70%, or 50%-60%, or by at least 2-fold, at least 5-fold, at least 10-fold, at least 20-fold, at least 50-fold, at least 100-fold, or at least 1000-fold compared to a control.

In some embodiments, an agent, or a combination of agents, is administered in an amount effective for treating, e.g., improving the symptoms of an inflammatory lung disease such as COPD by at least 20%-100%, 20%-90%, 20%-80%, 20%-70%, 20%-60%, 20%-50%, 30%-100%, 30%-90%, 30%-80%, 30%-70%, 30%-60%, 30%-50%, 40%-100%, 40%-90%, 40%-80%, 40%-70%, 40%-60%, 40%-50%, 50%-100%, 50%-90%, 50%-80%, 50%-70%, or 50%-60%, or by at least 2-fold, at least 5-fold, at least 10-fold, at least 20-fold, at least 50-fold, at least 100-fold, or at least 1000-fold compared to a control.

### Viral Infection

A subject may be, for example, a human subject. Other non-human subjects are also contemplated herein, for example, a livestock animal such as a cow, a sheep, a goat, a poultry, or a pig. Other non-human mammals subject to viral infection, such as respiratory virus infection, are also contemplated herein.

A subject, in some embodiments, is infected with a virus (e.g., a respiratory virus) or at risk of viral infection (e.g., a respiratory virus infection). A subject is considered “at risk of viral infection” if the subject is, for example, immunocompromised, a child (e.g., under the age of 18 years), an elderly person (e.g., over the age of 65 years), or has been in contact with or plans to be in contact with another person who is infected with a virus. In some embodiments, the subject is immunocompromised. In some embodiments, the subject is a child. In other embodiments, the subject is an elderly person.

In some embodiments, a subject has been exposed to a virus, such as a respiratory virus. Exposure to a virus includes indirect or direct contact with the virus. For example, a subject may be considered exposed to a virus if the subject was in the presence of another subject who has been infected with the virus. A subject “exposed to” a virus may also be “suspected of having” a viral infection. In some embodiments, a subject is infected with (and diagnosed with) a virus.

Non-limiting examples of respiratory viruses include influenza viruses (e.g., influenza A/Hong Kong/8/68 (H3N2), A/WSN/33 (H1N1), or influenza A/Avian Influenza (H5N1)), coronaviruses (e.g., betacoronavirus, e.g., MERS-CoV, SARS-CoV, or SARS-CoV-2), rhinoviruses, enteroviruses, parainfluenza viruses, metapneumoviruses, respiratory syncytial viruses, adenoviruses, and bocaviruses. Other virus and thus other viral infections are contemplated herein.

In some embodiments, a virus is an influenza virus. Influenza virus infects hosts such as humans and livestock animals (e.g., cattle, sheep, goat, poultry, or pig). Infection can result in global pandemics as the virus spreads among hosts who are contagious but have not yet developed symptoms of infection. Influenza virus primarily infects cells of the airway (e.g., lung epithelial, airway epithelial, and/or alveoli) before spreading throughout the body. The symptoms of influenza virus infection include, for example, congestion, cough, sore throat, fever, chills, aches, and fatigue, and typically appear two days after exposure to the virus and last less than a week. In more severe cases, complications of influenza virus infection can lead to pneumonia, secondary bacterial pneumonia, sinus infection, and worsening of previous health problems including asthma or heart failure. In the most severe cases, influenza virus infection can lead to death, particularly in young children, the elderly, and immunosuppressed subjects.

There are four types of influenza viruses: A, B, C and D. Human influenza A and B viruses cause seasonal epidemics of disease almost every winter in the United States. The emergence of a new and very different influenza A virus to infect people can cause an influenza pandemic. Influenza type C infections generally cause a mild respiratory illness and are not thought to cause epidemics. Influenza D viruses primarily affect cattle and are not known to infect or cause illness in people. Influenza A viruses are divided into subtypes based on two proteins on the surface of the virus: the hemagglutinin (H) and the neuraminidase (N). There are 18 different hemagglutinin subtypes and 11 different neuraminidase subtypes (H1 through H18 and N1 through N11 respectively). Influenza A viruses can be further broken down into different strains. Current subtypes of influenza A viruses found in people are influenza A (H1N1) and influenza A (H3N2) viruses. In the spring of 2009, a new influenza A (H1N1) virus (CDC 2009 H1N1 Flu website) emerged to cause illness in people. This virus was very different from the human influenza A (H1N1) viruses circulating at that time. The new virus caused the first influenza pandemic in more than 40 years. That virus (often called “2009 H1N1”) has now replaced the H1N1 virus that was previously circulating in humans. Herein, “H1N1” refers to any H1N1 virus circulating in humans. In some embodiments, Influenza A viruses can be influenza A/Hong Kong/8/68 (H3N2), A/WSN/33 (H1N1), or influenza A/Avian Influenza (H5N1), for example. Influenza B viruses are not divided into subtypes but can be further broken down into lineages and strains. Currently circulating influenza B viruses belong to one of two lineages: B/Yamagata and B/Victoria. See, e.g., cdc.gov/flu/about/viruses/types.htm (Centers for Disease Control and Prevention website).

An influenza virus infection as provided herein may be caused by any strain of influenza virus. In some embodiments, the influenza virus is an influenza type A virus, an influenza type B virus, or an influenza type C virus. In some embodiments, an influenza A strain is selected from the following subtypes: H1N1, H1N2, H1N3, H1N8, H1N9, H2N2, H2N3, H2N8, H3N1, H3N2, H3N8, H4N2, H4N4, H4N6, H4N8, H5N1, H5N2, H5N3, H5N6, H5N8, H5N9, H6N1, H6N2, H6N4, H6N5, H6N6, H6N8, H7N1, H7N2, H7N3, H7N7, H7N8, H7N9, H8N4, H9N1, H9N2, H9N5, H9N8, H10N3, H10N4, H10N7, H10N8, H10N9, H11N2, H11N6, H11N9, H12N1, H12N3, H12N5, H13N6, H13N8, H14N5, H15N2, H15N8, H16N3, H17N10, and H18N11. In some embodiments, the strain of influenza virus is an influenza A (H1N1) strain. In some embodiments, the strain of influenza virus is an influenza A (H3N2) strain. In some embodiments, the strain of influenza virus is an influenza A (H5N1) strain. Non-limiting examples of particular strains of influenza virus include influenza A/WSN/33 (H1N1), influenza A/Hong Kong/8/68 (H3N2), and influenza A/Avian Influenza (H5N1), influenza A/Netherlands/602/2009 (H1N1), and influenza A/Panama/2007/99 (H3N2).

In some embodiments, a virus is a coronavirus. Coronaviruses (CoV) are a large family of zoonotic viruses that are transmitted between animals and people, causing illness ranging from the common cold to more severe diseases such as Middle East Respiratory Syndrome (MERS-CoV) and Severe Acute Respiratory Syndrome (SARS-CoV). Other non-limiting examples of coronaviruses include coronavirus 229E and NL63, which are common human alpha coronaviruses, and OC43 and HKU1, which are common human beta coronaviruses. In some embodiments, the methods and composition provided herein are used to inhibit pathogenesis of an alpha coronavirus. In some embodiments, the methods and composition provided herein are used to inhibit pathogenesis of a beta coronavirus. Several known coronaviruses are circulating in animals that have not yet infected humans.

Common signs of coronavirus infection include respiratory symptoms, fever, cough, shortness of breath, and breathing difficulties. In more severe cases, infection can cause pneumonia, severe acute respiratory syndrome, kidney failure, and even death. On Feb. 11, 2020 the World Health Organization (WHO) announced an official name for the disease that is causing the 2019 novel coronavirus outbreak, first identified in Wuhan City, Hubei Province, China—“coronavirus disease 2019”, abbreviated as “COVID-19.” In COVID-19, ‘CO’ stands for ‘corona,’ VP for ‘virus,’ and ‘D’ for disease. Formerly, this disease was referred to as “2019 novel coronavirus” or “2019-nCoV.” In some embodiments, the coronavirus infection being inhibited is COVID-19, also referred to as SARS-CoV2.

In some embodiments, a virus is a rhinovirus. Rhinovirus, which belongs to the genus Enterovirus in the family Picornaviridae, is the most common viral infectious agent in humans and is the predominant cause of the common cold. The three species of rhinovirus (rhinovirus A, rhinovirus B, and rhinovirus C) include around 160 recognized types of human rhinovirus that differ according to their surface proteins (serotypes). Common signs of rhinovirus include runny nose, sneezing, sore throat, headache, cough, body aches, mild fever, ear infections, sinus infections, and lung problems such as bronchiolitis and pneumonia.

Without wishing to be bound by any theory, rhinovirus A and rhinovirus B use “major” ICAM-1 (Inter-Cellular Adhesion Molecule 1), also known as CD54 (Cluster of Differentiation 54), as receptors on respiratory epithelial cells. Some subgroups under rhinovirus A and rhinovirus B uses the “minor” LDL receptor. Rhinovirus C uses cadherin-related family member 3 (CDHR3) to mediate cellular entry. As the virus replicates and spreads, infected cells release distress signals known as chemokines and cytokines (which in turn activate inflammatory mediators). Cell lysis occurs at the upper respiratory epithelium.

In some embodiments, a virus is an enterovirus. Enterovirus is a genus of positive-sense single-stranded RNA viruses associated with several human and mammalian diseases. Enteroviruses can be classified based on the genotyping of VP1 capsid region such as EV-D68, EV-B69, EV-D70, EV-A71. Without wishing to be bound by any theory, EV-D68 can cause mild to severe respiratory illness. For more severe cases, difficulty breathing, wheezing or problems catching one's breath may occur.

The virus affects millions of people worldwide each year and is often found in the respiratory secretions such as saliva, sputum, or nasal mucus and stool of an infected person. Poliovirus, including PV-1, PV-2, and PV-3, can cause poliomyelitis, which is the most significant disease that can be caused by enterovirus. Common signs of poliovirus include mild respiratory illness (the common cold), hand, foot and mouth disease, acute hemorrhagic conjunctivitis, aseptic meningitis, myocarditis, severe neonatal sepsis-like disease, acute flaccid paralysis, and the related acute flaccid myelitis. In some embodiments, enterovirus includes non-polio enteroviruses such as Coxsackie A viruses, Coxsackie B viruses, echoviruses, and other enteroviruses. In some embodiments, enterovirus includes any serotypes that contribute to respiratory infections.

In some embodiments, a virus is a parainfluenza virus. Parainfluenza virus, or human parainfluenza virus, causes human parainfluenza. Parainfluenza virus comprises four distinct single-stranded RNA viruses belonging to the Paramyxoviridae family. HPIVs remain the second main cause of hospitalization in children under 5 years of age suffering from a respiratory illness. Parainfluenza virus can be classified as human parainfluenza virus type 1 (HPIV-1), human parainfluenza virus type 2 (HPIV-2), human parainfluenza virus type 3 (HPIV-3), and human parainfluenza virus type 4 (HPIV-4). HPIVs can be further categorized as respirovirus (HPIV-1 and HPIV-3) and rubulavirus (HPIV-2 and HPIV-4).

Common signs of parainfluenza virus include upper respiratory illness such as fever, runny nose, or cough, lower respiratory illness such as croup (infection of the vocal cords (larynx), windpipe (trachea) and bronchial tubes (bronchi)), bronchitis (infection of the main air passages that connect the windpipe to the lungs), bronchiolitis (infection in the smallest air passages in the lungs), or pneumonia (an infection of the lungs), and other illness such as sore throat, sneezing, wheezing, ear pain, irritability, or decreased appetite. In some embodiments, different types of parainfluenza virus may cause different symptoms. For example, HPIV-1 and HPIV-2 can cause croup, with HPIV-1 most often identified as the cause in children. Both can also cause upper and lower respiratory illness, and cold-like symptoms. HPIV-3 can cause bronchiolitis, bronchitis, and pneumonia. HPIV-4 may be associated with mild to severe respiratory illnesses.

In some embodiments, a virus is a metapneumovirus. Metapneumovirus, also called human metapneumovirus (HMPV), is a negative-sense single-stranded RNA virus of the family Pneumoviridae and is closely related to the Avian metapneumovirus (AMPV) subgroup C. HMPV infects airway epithelial cells in the nose and lung. Without wishing to be bound by any theory, HMPV attaches to the target cell via the glycoprotein (G) protein interactions with heparan sulfate and other glycosaminoglycans. The HMPV fusion (F) protein encodes an RGD (Arg-Gly-Asp) motif that engages RGD-binding integrins as cellular receptors, and then mediates fusion of the cell membrane and viral envelope in a pH-independent fashion. HMPV can cause upper and lower respiratory disease in people of all ages, especially among young children, older adults, and people with weakened immune systems. HMPV is associated with 5% to 40% of respiratory tract infections in hospitalized and outpatient children. Signs of HMPV infection includes cough, fever, nasal congestion, and shortness of breath. Clinical symptoms of HMPV infection may progress to bronchitis or pneumonia and are similar to other viruses that cause upper and lower respiratory infections.

In some embodiments, a virus is a respiratory syncytial virus. Respiratory syncytial virus (RSV), or human respiratory syncytial virus and human orthopneumovirus, is a common, contagious virus that causes infections of the respiratory tract. RSV is a negative-sense, single-stranded RNA virus. Without wishing to be bound by any theory, RSV is divided into two antigenic subtypes, subtype A and subtype B, based on the reactivity of the F and G surface proteins to monoclonal antibodies. The subtypes tend to circulate simultaneously within local epidemics, although subtype A tends to be more prevalent. RSV subtype A (RSVA) is thought to be more virulent than RSV subtype B (RSVB), with higher viral loads and faster transmission time.

RSV was discovered in 1956 and has since been recognized as one of the most common causes of childhood illness. It causes annual outbreaks of respiratory illnesses in all age groups. RSV infection can present with a wide variety of signs and symptoms that range from mild upper respiratory tract infections (URTI) to severe and potentially life-threatening lower respiratory tract infections (LRTI) requiring hospitalization and mechanical ventilation. While RSV can cause respiratory tract infections in people of all ages and is among the most common childhood infections, its presentation often varies between age groups and immune status. Reinfection is common throughout life, but infants and the elderly remain at highest risk for symptomatic infection. Signs of RSV infection can include runny nose, decrease in appetite, coughing, sneezing, fever, and wheezing. Severe infections can lead to bronchiolitis and pneumonia. RSV can also make chronic health problems worse such as people with asthma and people with congestive heart failure.

In some embodiments, a virus is an adenovirus. Adenovirus belongs to the family of Adenoviridae, which is a medium-sized (90-100 nm), nonenveloped (without an outer lipid bilayer) virus with an icosahedral nucleocapsid containing a double stranded DNA genome. Adenovirus can be classified as 57 human adenovirus types (HAdV-1 to 57) in seven species (Human adenovirus A to G). without wishing to be bound by any theory, different types or serotypes of adenovirus are associated with different conditions. For example, HAdV-B and C can be associated with respiratory disease. HAdV-B and D can be associated with conjunctivitis. In some examples, adenovirus types 3, 4 and 7 are most commonly associated with acute respiratory illness. In some examples, adenovirus type 7 has been associated with more severe outcomes than other adenovirus types, particularly in people with weakened immune systems. In some examples, adenovirus types 8, 19, 37, 53, and 54 can cause epidemic keratoconjunctivitis. Signs of adenovirus infection include common cold or flu-like symptoms such as fever, sore throat, acute bronchitis, pneumonia, pink eye (conjunctivitis), acute gastroenteritis (inflammation of the stomach or intestines causing diarrhea, vomiting, nausea and stomach pain), bladder inflammation or infection, and neurologic disease.

In some embodiments, a virus is a bocavirus. Bocavirus (HBoV), also called human bocavirus, which belongs to the genus Bocaparvovirus of virus family Parvoviridae and is a small (20 nm), non-enveloped virus. It is a new viral genus that was discovered in 2005 in upper respiratory secretions from acutely ill children. Without wishing to be bound by any theory, there are four human genotypes of BoV, which include type 1 to 4. HBoV1 and HBoV3 (and gorilla bocaparvovirus) are members of species Primate bocaparvovirus 1 whereas viruses HBoV2 and HBoV4 belong to species Primate bocaparvovirus 2. HBoV1 is strongly implicated in causing some cases of lower respiratory tract infection, especially in young children, and several of the viruses have been linked to gastroenteritis. Signs of bocavirus infection include acute respiratory tract infections, cough, wheezing, fever, cyanosis, runny nose, and diarrhea.

### Inhibition of Other Inflammatory Conditions

In some embodiments, a subject is diagnosed with acute respiratory distress syndrome (ARDS). ARDS causes fluid to leak into the lungs, making it difficult to get oxygen into the bloodstream. Without wishing to be bound by any theory, in the early stages of ARDS, fluid from the smallest blood vessels in the lungs starts to leak into the alveoli, which are the tiny air sacs where oxygen exchange takes place. The lungs become smaller and stiffer which leads to having difficulty of breathing and the amount of oxygen in the blood falls (“hypoxemia”). As the body becomes starved for oxygen, the brain and other tissues can be harmed and leads to organ failure.

In some embodiments, a subject has been exposed to harmful conditions that result in ARDS. For example, a subject may have sepsis or inhale high concentrations of smoke or chemical fumes. For example, a subject may infections or trauma. For example, a subject may have pneumonia. For example, a subject may have trauma to the head. For example, a subject may undergo blood transfusions. For example, a subject may have been infected with any of the respiratory viruses as disclosed herein.

In some embodiments, a subject is diagnosed with an inflammatory lung disease. Non-limiting examples of inflammatory lung disease include asthma, pulmonary fibrosis, and interstitial lung disease. The inflammatory lung disease may be, for example, a chronic inflammatory lung disease, such as Chronic Obstructive Pulmonary Disease (COPD). In some embodiments, an inflammatory lung disease is any lung disease that is contributed by either acute or chronic inflammations.

Chronic obstructive pulmonary disease (COPD) is a chronic inflammatory lung disease that causes obstructed airflow from the lungs and breathing-related problems. Symptoms include shortness of breath, cough, excess phlegm, mucus, or sputum production, chest tightness, lack of energy, swelling in ankles, feet or legs, and wheezing. COPD can be caused by long-term exposure to irritating gases or particulate matter, most often from cigarette smoke. Other factor such as air pollutants, genetic factors, and respiratory infections may contribute to the onset of COPD. Emphysema and chronic bronchitis are the two most common conditions that contribute to COPD. Chronic bronchitis is characterized by daily cough and mucus (sputum) production. Emphysema is a condition in which the alveoli at the end of bronchioles of the lungs are destroyed as a result of damaging exposure to cigarette smoke and other irritating gases and particulate matter.

In some embodiments, the present disclosure, at least in part, identifies S100A7 as a biomarker for COPD, that can be subject to the RAGE pathway inhibitor.

In some embodiments, a subject requires use of a respiratory ventilator, which can exert cyclic mechanical strain on the lungs. In such subject, RAGE pathway inhibitors may be used to suppress inflammation due to this cyclic mechanical strain on the lungs.

### Examples

### Example 1. Evaluation of Gene Expression of the Alveolar Epithelial Cells

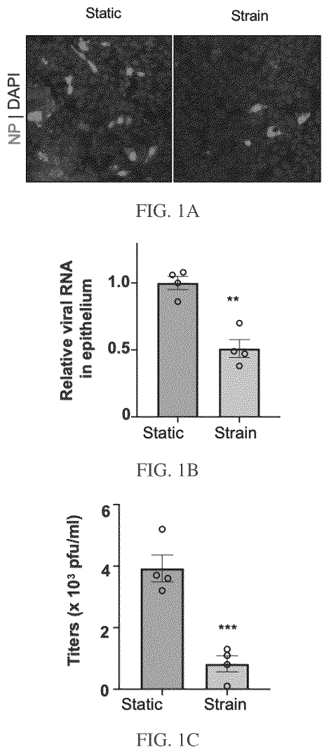

The alveolar epithelial cells that line the air sacs of the lung experience cyclic mechanical deformations during breathing motions. To study the underlying mechanisms, it was discovered that breathing motions inhibit infection of alveolar epithelial cells by H3N2 influenza virus (FIG. 1) in human Organ Chip models of the human lung alveolus (Alveolus Chip).

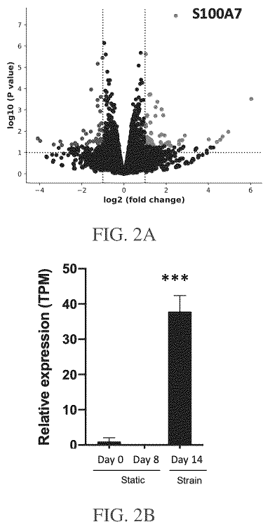

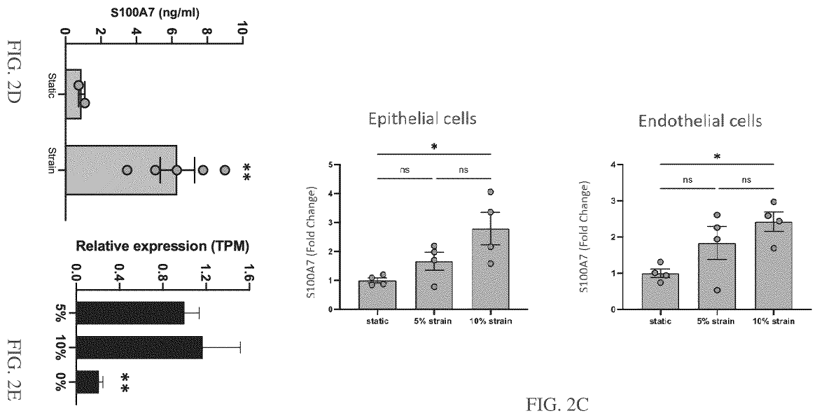

Changes of gene expression induced in the epithelial cells lining the human Alveolus Chip when exposed to cyclic mechanical strains using RNA-seq were analyzed. The top upregulated gene was S100A7 (also known as psoriasin) (FIG. 2A), which belongs to the S100 protein family. Without wishing to be bound by any theory, in humans, the S100 protein family is composed of 21 members that exhibit a high degree of structural similarity but are not functionally interchangeable. This family of proteins modulates cellular responses by acting both as intracellular Ca²⁺ sensors and as extracellular factors that bins to various membrane receptors. S100A7, along with S100A6, S100A8, S100A9, and S100A12, bind to the receptor for advanced glycation end-products (RAGE) and thereby, promote inflammation. Type I lung alveolar epithelial cells exhibit the highest expression of RAGE in humans. Therefore, it was speculated that mechanical strain may alter inflammatory responses to viral infection by inducing S100A7 expression and promoting its binding to RAGE.

A 35-fold increase of S100A7 gene expression in Lung Alveolus Chips in response to exposure to 5% cyclic strain at 0.25 Hz for 4 days was found (FIG. 2B). Mechanical strain induced the expression of S100A7 in epithelial cells and in endothelial cells (FIG. 2C). Strain-induced S100A7 protein was secreted as measured in the apical washes from the chip using an ELISA (FIG. 2D). The effect of mechanical strains on S100A7 expression was reversible, which means that maintaining 5% strain or increasing to 10% strain did not affect S100A7 mRNA level, while switching to 0% strain (static conditions) significantly decreased its expression (FIG. 2E).

In summary, the RAGE ligands, S100A7, was major effector that can be induced by breathing motions in the lung and thereby suppress viral infection.

### Example 2. The Expression of S100A7 and Other 5100 Family Genes in Respiratory Pathogenesis

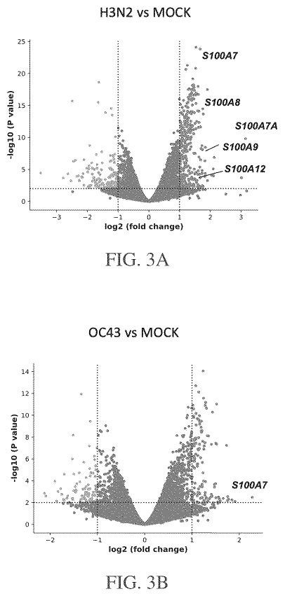

S100A7 was first found in psoriatic skin where it acted as an anti-microbial peptide and protects human skin from Escherichia coli infection. The role of S100A7 in viral infection, however, was less clear. To characterize the host response to influenza infection in the Human Alveolus Chips, S100A7, S100A7A, S100A8, S100A9, and S100A12 were found to be upregulated upon H3N2 influenza virus infection in the alveolar epithelial cells (FIG. 3A). S100A7 expression also increased after infection with OC43 coronavirus (FIG. 3B) that caused the common cold. Without wishing to be bound by any theory, in a study of host responses in COVID-19 patients, massive amounts of plasma S100A8 and S100A9 were found in severe cases, which correlated uncontrolled inflammation in these patients. Taken together, these data indicate that multiple different types of respiratory viruses potently induce S100 family proteins.

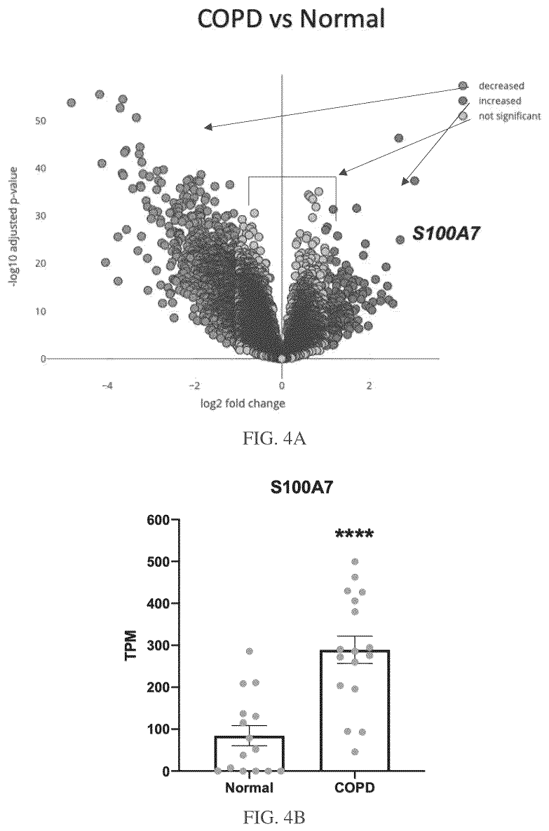

Chronic obstructive pulmonary disease (COPD) is a chronic inflammatory lung disease that is characterized by disrupted alveolar walls and elevated lung compliance. S100A7 expression levels in RNAseq data of lung airway epithelial cells from 5 healthy donors and 5 COPD patients were compared. It was surprisingly found that S100A7 was a top upregulated gene in COPD patients (FIG. 4A). The transcripts per million (TPM) for S100A7 was 3-fold higher in COPD patients compared with healthy control (FIG. 4B). This suggests a role of S100A7 as a novel biomarker in COPD pathogenesis, which was characterized by both hyperinflammation and increased lung mechanical strain.

### Example 3. Effects of Inhibitors of S100A7 on Influenza-Induced Host Inflammatory Response

S100A7 and other members of the S100A7 family protein signal through the RAGE receptor. It was hypothesized that inhibitors of RAGE may decrease the host inflammatory response in settings where S100 family proteins are increased. No RAGE pathway inhibitors have been FDA-approved. However, azeliragon (TTP488, TransTech Pharma), is currently in Phase 3 clinical trials in patients with Alzheimer Disease.

Azeliragon

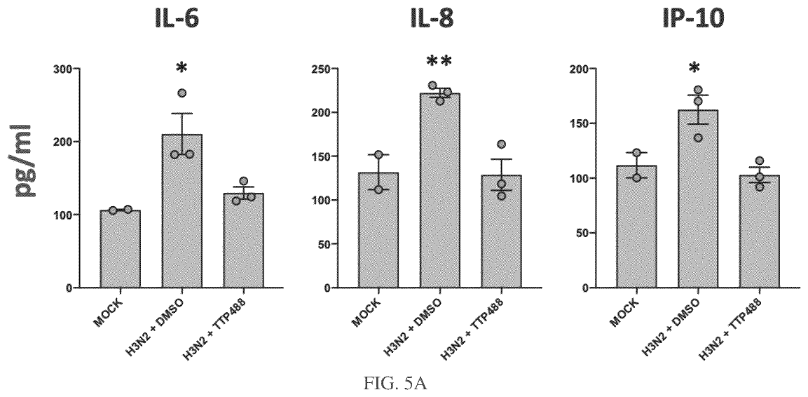

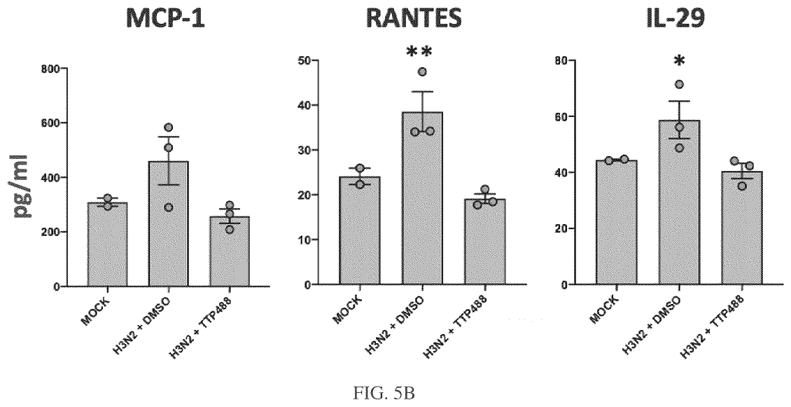

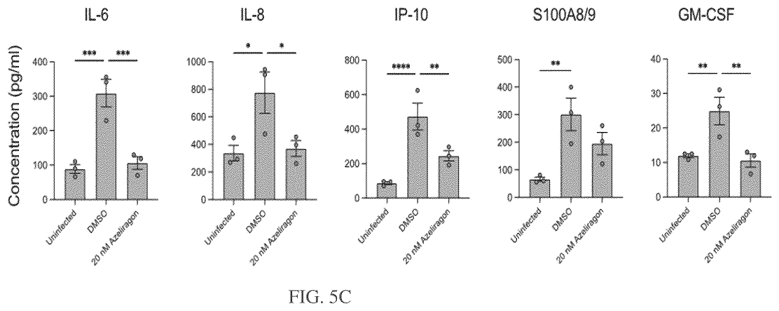

It was found that azeliragon significantly blocked the induction of a number of cytokines, including IL-6, IL-8, IP-10, MCP-1, RANTES, and IL-29, in Human Alveolus Chips infected with H3N2 influenza virus when azeliragon was administered at a clinically relevant dose (100 nM), which represented its Cmax in human blood (FIGS. 5A-5B). Azeliragon was similarly effective in blocking the induction of cytokines (IL-6, IL-8, IP-10, and Granulocyte-macrophage colony-stimulating factor (GM-CSF)) at a lower dose (20 nM azeliragon) in Human Alveolus Chips infected with H3N2 influenza virus (FIG. 5C). It was found that azeliragon or its derivatives suppressed inflammation associated with infection by various types of respiratory viruses, including various influenza and corona virus strains (including SARS-CoV-2).

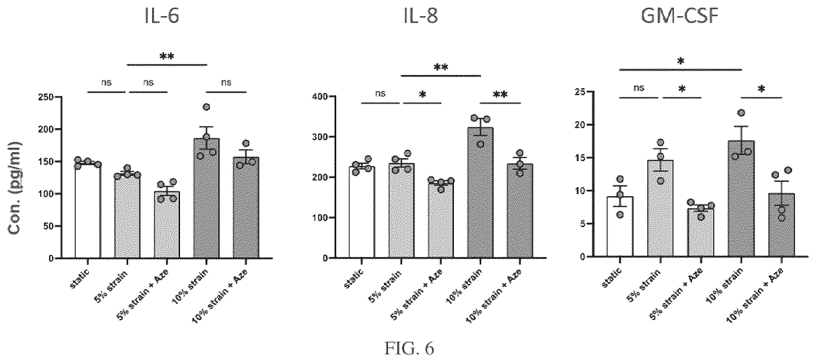

Hyper-physiological mechanical strain induces inflammation (also called biotrauma), which can be caused in the lung by ventilator-induced injury, breathing compressed air, and lung disorders, such as chronic obstructive pulmonary disease. It was shown that azeliragon inhibits such inflammation, supporting the potential application of this drug for patients who are at risk of ventilator-induced lung injury (such as COVID patients on ventilator). DMSO control or 100 nM azeliragon were perfused for 48 hours in the presence of 0% strain, 5% strain, or 10% strain; vascular outlet was collected for Luminex assay (FIG. 6).

FPS-ZM1

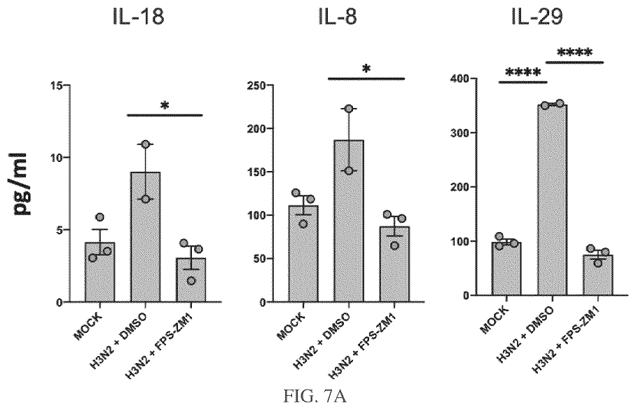

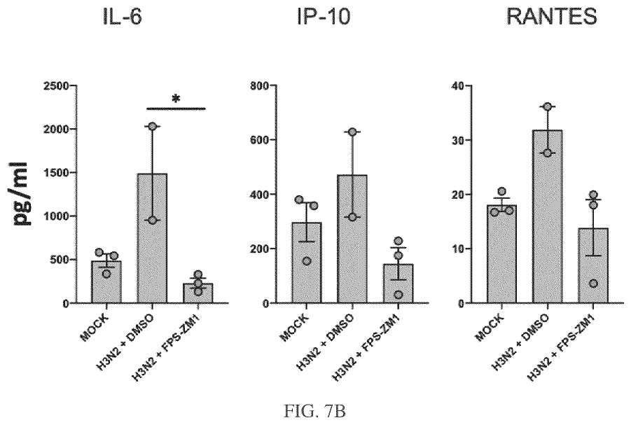

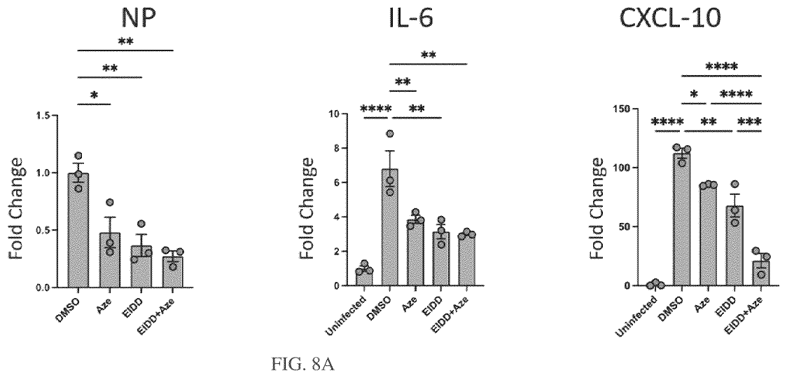

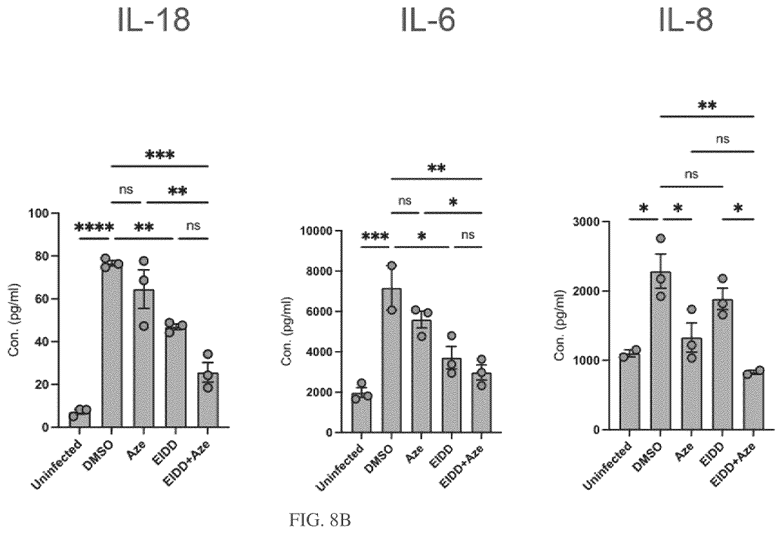

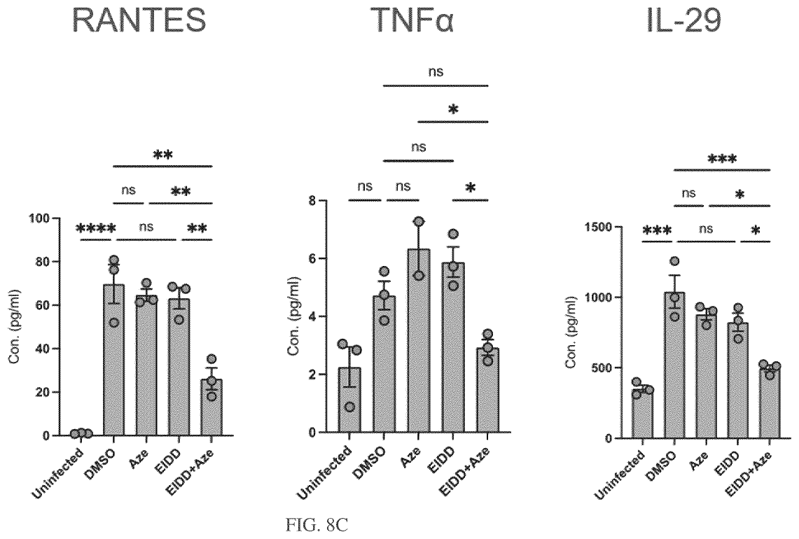

Another RAGE pathway inhibitor, FPS-ZM1, also inhibited virus-mediated secretion of inflammatory cytokines (FIGS. 7A-7B).

Soluble RAGE, siRNA or CRISPR Cas9 against RAGE may also be used. These drugs may be used in combination with antivirals as well as inducers of host protective responses (e.g., type I interferons). In addition, since S100A7 was a top upregulated gene in COPD patients, inhibitors of the S100A7/RAGE pathway may be used as a treatment to suppress inflammation in COPD patients. As inflammation and ARDS (acute respiratory distress syndrome) are observed in patients on respiratory ventilators that exert cyclic mechanical strain on lung, RAGE pathway inhibitors may also be useful to suppress inflammation in these conditions. Thus, blocking S100A7-RAGE pathway can inhibit host viral inflammatory response in the lung, although it did not alter viral load.

### Example 4. Combination Therapies

The effects of the combination azeliragon with several antiviral agents on viral load and host inflammatory response were assessed using the Alveolus Chip model.

Azeliragon and Molnupiravir

The first combination tested was azeliragon and molnupiravir. Molnupiravir is an orally bioavailable ribonucleoside analog with broad-spectrum antiviral activity against various unrelated RNA viruses including influenza, Ebola, CoV, Venezuelan equine encephalitis virus (VEEV), SARS-CoV-2, MERS-CoV, SARS-CoV, and related zoonotic group 2b or 2c bat-CoVs, as well as increased potency against a CoV bearing resistance mutations to the nucleoside analog inhibitor remdesivir. It was also found that while the combination of azeliragon and the antiviral molnupiravir had no synergistic effect on viral load, surprisingly, the two agents were synergistic in suppressing inflammatory cytokine production in epithelial cells following H3N2 infection (FIGS. 7A-7C).

Azeliragon and Favipiravir

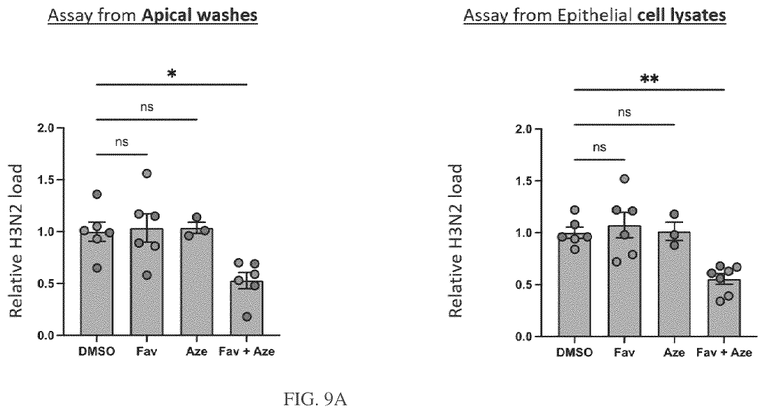

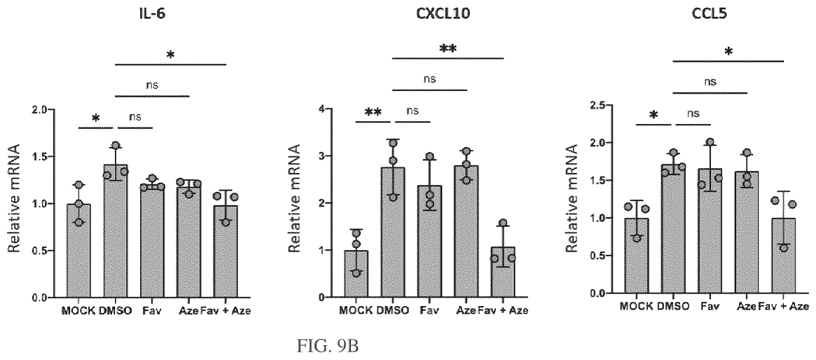

The second combination tested was azeliragon and favipiravir. Favipiravir is an approved broad-spectrum anti-influenza drug that inhibits RNA polymerase. Favipiravir also inhibits infection of SARS-CoV-2 and is in multiple clinical trials against COVID. Surprisingly, the combination of azeliragon and favipiravir decreased viral load (FIG. 9A) and decrease the host inflammatory response (FIG. 9B).

### REFERENCES

1 Tomazini, B. M. et al. Effect of Dexamethasone on Days Alive and Ventilator-Free in Patients With Moderate or Severe Acute Respiratory Distress Syndrome and COVID-19: The CoDEX Randomized Clinical Trial. JAMA: the journal of the American Medical Association 324, 1307-1316, doi:10.1001/jama.2020.17021 (2020).
2 Bresnick, A. R., Weber, D. J. & Zimmer, D. B. S100 proteins in cancer. Nat Rev Cancer 15, 96-109, doi:10.1038/nrc3893 (2015).
3 Hudson, B. I. & Lippman, M. E. Targeting RAGE Signaling in Inflammatory Disease. Annu Rev Med 69, 349-364, doi:10.1146/annurev-med-041316-085215 (2018).
4 Oczypok, E. A., Perkins, T. N. & Oury, T. D. All the “RAGE” in lung disease: The receptor for advanced glycation endproducts (RAGE) is a major mediator of pulmonary inflammatory responses. Paediatr Respir Rev 23, 40-49, doi:10.1016/j.prrv.2017.03.012 (2017).
5 Glaser, R. et al. Antimicrobial psoriasin (S100A7) protects human skin from Escherichia coli infection. Nat Immunol 6, 57-64, doi:10.1038/ni1142 (2005).
6 Silvin, A. et al. Elevated Calprotectin and Abnormal Myeloid Cell Subsets Discriminate Severe from Mild COVID-19. Cell, doi:10.1016/j.cell.2020.08.002 (2020).

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between and including the upper and lower ends of the range are specifically contemplated and described herein.

1. A method of treating a viral infection in a subject in need thereof, comprising administering to the subject a receptor for advanced glycation end-products (RAGE) pathway inhibitor.

2. The method of claim 1, wherein the subject is infected with a virus.

3. The method of claim 1, wherein the subject is at risk of viral infection.

4. The method of claim 1, wherein the viral infection is a respiratory virus infection.

5. The method of claim 1, wherein the respiratory virus is selected from the group consisting of: influenza viruses (e.g., influenza A/Hong Kong/8/68 (H3N2), A/WSN/33 (H1N1), or influenza A/Avian Influenza (H5N1)), coronaviruses (e.g., betacoronavirus, e.g., SARS-CoV-2), rhinoviruses, enteroviruses, parainfluenza viruses, metapneumoviruses, respiratory syncytial viruses, adenoviruses, bocaviruses, and common cold viruses.

6. The method of claim 1, wherein the subject has a symptom one or more symptom(s) of a viral infection.

7. The method of claim 6, wherein the symptom is related to aberrant inflammation.

8. The method of claim 6, wherein the symptom is inflammation in the lungs.

9. The method of claim 6, wherein the symptom is inflammation in an organ other than the lungs.

10. A method of treating acute respiratory distress syndrome (ARDS) or pulmonary barotrauma in a subject in need thereof, comprising administering to the subject a receptor for advanced glycation end-products (RAGE) pathway inhibitor.

11.-12. (canceled)

13. A method of treating an inflammatory disease in a subject in need thereof, comprising administering to the subject a receptor for advanced glycation end-products (RAGE) pathway inhibitor, wherein the subject is diagnosed with an inflammatory lung disease.

14.-44. (canceled)
